(19) United States
(12) Patent Application Publication
(10) Pub. No.: US 2018/0179250 A1
(43) Pub. Date: Jun. 28, 2018

(54) LIGAND IMMOBILIZATION METHOD

(71) Applicant: Kaneka Corporation, Osaka (JP)

(72) Inventors: Fuminori Konoike, Hyogo (JP); Tatsuya Honda, Hyogo (JP); Keiichi Karasugi, Hyogo (JP)

(73) Assignee: Kaneka Corporation, Osaka (JP)

(21) Appl. No.: 15/905,635

(22) Filed: Feb. 26, 2018

Related U.S. Application Data
(63) Continuation of application No. PCT/JP2016/075000, filed on Aug. 26, 2016.

(30) Foreign Application Priority Data
Aug. 27, 2015 (JP) 2015-168066

(51) Int. Cl. C07K 1/22; C07K 17/12; C07K 17/10; C07K 17/08; C07K 17/14; B01J 20/24; B01J 20/26; B01J 20/32; B01D 15/38

(52) U.S. Cl. CPC C07K 1/22; C07K 17/12; C07K 17/10; C07K 17/08; C07K 17/14; B01D 15/38; B01J 20/265; B01J 20/3208; B01J 20/3219; B01J 20/3278; B01J 20/24

(57) ABSTRACT

A method for immobilizing a ligand on a formyl group-containing insoluble base material includes producing an imine by mixing the ligand and the formyl group-containing insoluble base material, and reducing the imine by using a borane complex, wherein the ligand comprises an amino group and has a specific affinity for a target compound, and wherein the borane complex has a Lewis base ligand having pKₐ of 6.5 or less.

### TECHNICAL FIELD

One or more embodiments of the present invention relate to a method for firmly immobilizing a ligand on a formyl group-containing insoluble base material. According to one or more embodiments of the present invention, an adsorbent from which the ligand is remarkably prevented from being leaked can be produced.

### BACKGROUND

An availability of a physiologically active substance such as a peptide having a specific affinity for a certain compound and a substrate of an enzyme is enhanced by immobilizing on an insoluble base material, since a substance which interacts with the immobilized physiologically active substance can be recovered and detected. For example, according to an affinity chromatography, only a target compound can be efficiently recovered from a mixture by immobilizing a physiologically active substance which specifically binds to the target compound on insoluble porous beads as a ligand. As an industrial application of affinity chromatography, a separation of an immunoglobulin using an immobilized protein and a separation of an antigen using an immobilized antibody are exemplified.

As an embodiment to immobilize a ligand on an insoluble base material, it is very important for an industrial application to immobilize the ligand by a firm covalent bond in order to reduce a leakage of the immobilized ligand. In addition, a condition of an immobilized ligand is also important. For example, it may be possible that a ligand is immobilized while an activity thereof is maintained.

As a method for immobilizing a ligand on an insoluble base material, a method in which an epoxy group is introduced into an insoluble base material and a thiol group or an amino group in the ligand is reacted with the epoxy group is exemplified. However, when a ligand is a protein, a side chain thiol group of a cysteine residue is involved in a stabilization of a higher-order structure of a protein through a formation of a disulfide bond in some cases. If such a disulfide bond is reduced to be thiol groups, a higher-order structure of a protein may be changed and an affinity for a target compound may be decreased. In addition, a lower yield may be problematic for a method in which an amino group is reacted with an epoxy group.

Accordingly, a method for immobilizing a ligand having an amino group on an insoluble base material by a reductive amination reaction has been developed (Patent Document 1). In the method, a formyl group is introduced into a base material, the formyl group is reacted with a ligand to obtain an imine, and the imino group is reduced to be a stable amino group.

Patent Document 1: WO 2010/064437

As described above, a method for producing an adsorbent by immobilizing a ligand having an amino group on an insoluble base material with a reductive amination reaction has been developed.

On the one hand, the inventors found that even when an adsorbent obtained by such a method is used, a ligand is detached from insoluble porous beads, for example, during an affinity chromatography. Such a detached ligand is mixed in an eluate and finally in a target compound. In addition, a stability of an instrument performance is impaired due to a detachment of a ligand immobilized on a sensor of an analytical instrument.

Accordingly, one or more embodiments of the present invention provide a method for firmly immobilizing a ligand on a formyl group-containing insoluble base material by which method an adsorbent from which the ligand is remarkably prevented from being leaked can be produced. Also, one or more embodiments of the present invention provide a method for purifying a target compound using an adsorbent produced by the above-described method.

The inventors found that a detachment of a ligand is closely related to pKₐ of a Lewis base ligand of a borane complex used as a reducing agent.

### SUMMARY

Hereinafter, one or more embodiments of the present invention are described.

[1] A method for immobilizing a ligand on a formyl group-containing insoluble base material,

wherein the ligand has a specific affinity for a target compound and has an amino group,

comprising the steps of:

producing an imine by mixing the ligand and the formyl group-containing insoluble base material, and reducing the imine by using a borane complex, wherein the borane complex has a Lewis base ligand having pKₐ of 6.5 or less.

[2] The method according to the above [1], wherein the Lewis base ligand is a nitrogen-containing heterocyclic aromatic compound and/or an aromatic hydrocarbon compound having an amino group as a substituent.

[3] The method according to the above [1] or [2], wherein the ligand is a peptide.

[4] The method according to the above [3], wherein the ligand is an antibody affinity ligand.

[5] The method according to the above [4], wherein the antibody affinity ligand is Protein A, Protein G, Protein L or an analog thereof.

[6] The method according to any one of the above [1] to [5], wherein the formyl group-containing insoluble base material is essentially composed of at least one selected from the group consisting of a polysaccharide, a synthetic polymer and glass.

[7] The method according to any one of the above [1] to [5], wherein the formyl group-containing insoluble base material is essentially composed of cellulose or agarose.

[8] The method according to any one of the above [1] to [7], wherein a form of the formyl group-containing insoluble base material is a porous bead, a monolith or a porous membrane.

[9] A method for purifying the target compound, comprising the steps of:
producing an adsorbent by immobilizing the ligand on the formyl group-containing insoluble base material by the method according to any one of claims 1 to 8, and
contacting a mixture containing the target compound with the adsorbent to adsorb the target compound on the adsorbent.

In one or more embodiments of the present method, a ligand is immobilized on an insoluble base material more firmly in comparison with a conventional method; as a result, a leakage of the ligand is remarkably suppressed. One or more embodiments of the present invention may produce a specific adsorbent by which a highly pure target compound containing a lower amount of a mixed impurity can be obtained.

### BRIEF DESCRIPTION OF THE DRAWINGS

FIG. 1 is a graph to demonstrate the relation between pKₐ of a Lewis base ligand of a borane complex used as a reducing agent and a leakage amount of a ligand.

FIG. 2 is a graph to demonstrate the result of a ligand leakage amount test of various ligand-immobilized adsorbents.

### DETAILED DESCRIPTION OF THE EMBODIMENTS

Hereinafter, each step of one or more embodiments of the present invention is described.

1. Step of Introducing Formyl Group into Insoluble Base Material

When a formyl group-containing insoluble base material is available, for example, as a commercially available product, it is not needed to execute this step. On the one hand, when a formyl group-containing insoluble base material is not available, a formyl group may be introduced into an insoluble base material according to a conventional method.

An insoluble base material is not particularly restricted as long as the insoluble base material is insoluble in a solvent of a mixture containing a target compound, such as water, and can adsorb a target compound. For example, the insoluble base material is exemplified by porous beads used as a packing material for chromatography, a biosensor of an analysis instrument to detect a target compound, a monolith used for recovering and analyzing a target compound, a porous membrane used for recovering a target compound and removing a misplaced material, and a chip such as a protein microarray. As a biosensor of an analysis instrument, a sensor chip of an analysis instrument which utilizes surface plasmon resonance or biolayer interferometry is exemplified.

A raw material which constitutes the insoluble base material is not particularly restricted as long as the raw material is insoluble in water, and is exemplified by a polysaccharide such as cellulose, agarose, dextran, starch, pullulan, chitosan and chitin; a synthetic polymer such as poly(meth)acrylic acid, poly(meth)acrylate ester, polyacrylamide and polyvinyl alcohol, and a crosslinked synthetic polymer; and glass such as silica glass, borosilicate glass, optical glass and soda glass. In addition, a surface of a base material composed of a synthetic polymer which does not have a functional group, such as polystyrene and styrene-divinylbenzene copolymer, may be coated with a polymer material having a reactive functional group such as a hydroxy group. Such a coating polymer material is exemplified by a graft copolymer such as a copolymer of hydroxyethyl methacrylate or a monomer having a polyethylene oxide chain and other polymerizable monomer having a reactive functional group. Among the above examples, a polysaccharide and polyvinyl alcohol may be used, since an active group can be easily introduced on the surface of the base material.

A size of a porous bead as an insoluble base material may be appropriately adjusted, and may be 20 μm or more and 1000 μm or less as volume average particle diameter. When the volume average particle diameter is 20 μm or more, a back pressure may be kept lower when a column is filled with the beads. On the one hand, the volume average particle diameter is 1000 μm or less, a surface area is large and an amount of an adsorbed target compound amount may become large. The volume average particle diameter may be 30 μm or more, 40 μm or more, 50 μm or more, or 60 μm or more, and may be 250 μm or less, 125 μm or less, 100 μm or less, or 85 μm or less. The volume average particle diameter of porous beads can be determined by randomly selecting 100 porous beads and measuring the particle diameters thereof. A particle diameter of each porous bead can be measured by taking a microscope photograph of the porous bead, saving an electronic data of the photograph, and using a particle diameter measurement software such as “Image-Pro Plus” manufactured by Media Cybernetics. It may be possible that porous beads are crosslinked with a multifunctional compound by an ordinary method in order to improve the strength.

A monolith is a kind of a porous continuous structure and a sponge-like structure having a hole and a skeleton which forms the structure. A monolith is excellent in a mass transfer property and a pressure-flow speed property. By controlling sizes of a hole and skeleton, an adsorption efficiency and a separation efficiency of a target compound, a liquid permeability and a detection sensitivity can be improved. It can be confirmed whether a structure is continuously porous or not by observing a similar hole on various cross-section surfaces of the structure by using an electron scanning microscope.

For example, a porous membrane has a form such as a flat membrane, a hollow fiber and a depth filter structure.

A pore diameter of the insoluble base material having holes, such as a monolith and a porous membrane, may be appropriately adjusted depending on a target compound to be adsorbed and a flow rate, and may be adjusted to, for example, 1 nm or more and 10 μm or less. For example, a target compound is an antibody or an antibody fragment, a pore diameter may be 10 nm or more and 300 nm or less.

As a method for producing the insoluble base material from a raw material, a conventional method may be used. For example, in a case of porous beads, a solution or dispersion of a raw material polymer is dispersed in a fat and oil to be a droplet, and the droplet is contacted with a solvent which is miscible in the solvent of the solution or dispersion, such as an alcohol or an aqueous alcohol, to obtain a porous particle.

In order to introduce a formyl group into an insoluble base material, a functional group of a raw material which constitutes the insoluble base material or a coating material may be utilized. For example, a polysaccharide as the raw material has many hydroxy groups. An epoxy group can be introduced by reacting the hydroxy group with a halohydrin such as epichlorohydrin. Alternatively, when a polyepoxide compound is used, an unreacted epoxy group may be remained. A ring of an epoxy group is easily opened by an acidic aqueous solution or a basic aqueous solution. A ring-opened epoxy group is a 1,2-diol group, and a 1,2-diol group can be oxidized to be a formyl group by an oxidizing agent.

As an oxidizing agent to oxidize a hydroxy group to be a formyl group is exemplified by periodic acid and a periodate salt. A periodate salt is exemplified by sodium periodate and potassium periodate.

An amount of a formyl group of the formyl group-containing insoluble base material is not particularly restricted, and may be 0.5 μmol or more and 100 μmol or less per 1 mL of the formyl group-containing insoluble base material. When the formyl group amount per 1 mL of the formyl group-containing insoluble base material is 0.5 μmol or more, an affinity ligand may be efficiently immobilized; and further, when the formyl group-containing insoluble base material is used for an adsorbent, an adsorption amount of a target compound may be large. In addition, though the reason is not clear, when the formyl group amount per 1 mL of the formyl group-containing insoluble base material is 100 μmol or less, an adsorption amount of a target compound may be large amazingly. When a formyl group is introduced by using periodic acid and/or a periodate salt and the formyl group amount per 1 mL of the formyl group-containing insoluble base material is 100 μmol or less, the strength of the formyl group-containing insoluble base material may be high. The formyl group amount per 1 mL of the formyl group-containing insoluble base material may be 1 μmol or more, 1.5 μmol or more, or 2 μmol or more, and may be 50 μmol or less, 25 μmol or less, 10 μmol or less, or 7 μmol or less. For example, the formyl group amount can be adjusted by a time, temperature and a concentration of a formylating agent such as periodic acid and/or a periodate salt of a formylating reaction. A volume of the formyl group-containing insoluble base material as a base of the above-described formyl group amount is a volume of a whole structure containing a hole and a skeleton in the case of a monolith and a porous membrane, or is a tapping volume in the case of porous beads unless otherwise specified. A tapping volume means a volume measured after a slurry containing porous beads and reverse osmosis water is added into a measuring container and vibration is given to the container until a volume of the porous beads is not decreased any more.

The formyl group amount can be measured by adding a phenylhydrazine solution to the formyl group-containing insoluble base material, stirring the mixture at 40° C. for 1 hour, measuring an absorption spectrum of the supernatant after the reaction by using UV, and determining an amount of a reduced phenylhydrazine from a calibration curve of phenylhydrazine.

2. Step of Introducing Amino Group to Ligand

When a ligand has an amino group, it is not needed to execute this step. On the one hand, when a ligand does not have an amino group, an amino group is introduced to a ligand.

The ligand which should be bound to the insoluble base material in one or more embodiments of the present invention means a substance which can selectively bind to a target compound in an aggregation of certain molecules by a specific affinity for the target compound. The ligand has an affinity for a target compound, and is exemplified by a peptide, a polysaccharide, a substrate of an enzyme, and DNA. The term “peptide” in one or more embodiments of the present invention means a compound which is composed of two or more amino acids bound to each other through a peptide bond and which has a specific affinity for a target compound. Such a peptide is exemplified by a protein which has a specific affinity for a target compound, a subunit and a domain of a protein of which specific affinity for a target compound is maintained, and an antibody fragment such as a Fab region. The above protein is exemplified by a receptor protein which binds to a substrate compound, an antibody which binds to an antigen, and lectin which binds to a sugar chain.

A peptide usable as a ligand is exemplified by an antibody affinity ligand. Such an antibody affinity ligand is exemplified by Protein A, Protein G, Protein L, Protein H, Protein D, Protein Arp, Protein FcγR, an antibody-binding synthetic ligand, and an analog thereof. The term “analog of the antibody affinity ligand” in the present disclosure means a variant which has an amino acid sequence of the above-described Protein A or the like with deletion, substitution and/or addition of one or more amino acids and of which affinity for a target antibody or a fragment thereof is maintained or improved in comparison with natural Protein A or the like, or a subunit or a domain of which affinity for a target antibody or a fragment thereof is maintained. The upper limit of the variation number such as deletion in the above-described variant is dependent on the amino acid which constitutes an original peptide, and the variation number may be, for example, 20 or less, not more than 10 or 5, or not more than 3 or 2.

In the case of a substrate of an enzyme or a sugar chain, which does not have an amino group, an amino group is introduced thereto. It is easy for a person skilled in the art to transform a functional group in a substrate and a sugar chain to an amino group and to introduce an amino group by utilizing such a functional group. When there is an amino group at N-terminal only or there is not a sufficient amount of a side-chain amino group in a peptide to be a ligand, a basic amino acid such as lysine or a derivative thereof can be introduced in an optional position or an optional position can be substituted by a basic amino acid or a derivative thereof by a gene recombination technology or a synthesis technology. In addition, when there is not a usable amino group or there is an insufficient amount of a usable amino group in DNA or a sugar, an amino group can be introduced by a similar technology.

A compound targeted by the ligand in one or more embodiments of the present invention is not particularly restricted as long as the compound is an object to be purified or detected and the ligand can specifically bind to the compound. Such a compound is exemplified by an immunoglobulin G (IgG) and an immunoglobulin G derivative which binds to an antibody-binding synthetic ligand such as Protein A, Protein G, Protein L, Protein H, Protein D, Protein Arp and Protein FcγR; a glycoprotein, which binds to lectin; plasminogen, which binds to lysine; biotin, which binds to avidin; a protease, which binds to a protease inhibitor; a nucleotide-binding protein, which binds to triazine; src kinase, which binds to casein or tyrosine. An antibody fragment which can bind to each antibody-binding synthetic ligand is included in the above-described immunoglobulin G derivative.

3. Step of Reacting Ligand and Insoluble Base Material

In this step, the ligand which has a specific affinity for a target compound and which has an amino group is mixed with the formyl group-containing insoluble base material to form an imine from the amino group contained in the ligand and the formyl group contained in the insoluble base material.

It may be possible that the pH of a reaction mixture for imination reaction of the ligand and the insoluble base material is included in a range of 10.0 or more and less than 13.0, since an amount and/or a ratio of the immobilized amino group-containing ligand is large in the range. The pH can be measured using a pH meter which is subjected to three-point calibration using a standardized solutions having pH of 3 to 5, 6 to 7 and 9 to 10. The term “amount of introduced ligand” is synonymous with the term “amount of immobilized amount” in one or more embodiments of the present invention.

A solvent for the above-described imination reaction may be a buffer solution in terms of a stability of pH. A buffer solution usable in one or more embodiments of the present invention is not particularly restricted, and a conventionally-known buffer solution may be used.

A temperature for the above-described imination reaction may be appropriately adjusted, and may be adjusted to −10° C. or higher and 40° C. or lower. The reaction temperature of −10° C. or higher may be used in terms of a fluidity of the reaction mixture. When the reaction temperature is 40° C. or lower, the affinity ligand and the formyl group of the insoluble base material may be difficult to be deactivated. The reaction temperature may be −5° C. or higher, or 0° C. or higher, and may be 35° C. or lower, or 30° C. or lower.

A reaction time may be adjusted so that the ligand and the insoluble base material are sufficiently reacted, may be specifically determined by a preliminary experiment, and may be adjusted to, for example, 1 hour or more and 50 hours or less.

After the reaction, an ordinary aftertreatment may be conducted; however, it may be possible that the reaction mixture is directly subjected to the next step, since an imino group is relatively unstable.

4. Step of Reducing Imino Group

In this step, the imino group formed from the amino group of the ligand and the formyl group of the insoluble base material in the previous step is reduced using a borane complex which has a Lewis base ligand having pKₐ of 6.5 or less. It is presumed that by using such a borane complex, the imino group formed from the formyl group of the insoluble base material and the amino group of the ligand can be sufficiently reduced, the ligand can be immobilized on the insoluble base material, and a leakage of the ligand can be remarkably suppressed.

The above-described pKₐ is a −log value of an acid dissociation constant Kₐ of the above-described Lewis base in water at 25° C. The inventors experimentally found that an amount of a leaked ligand is precipitously increased while the pKₐ is changed from 6.2 to 6.6, but when the pKₐ is 6.5 or less, an amount of a leaked ligand can be remarkably suppressed. Specifically, an amount of a leaked ligand can be 50 ppm or less in the condition of Examples described later. The leakage amount may be 40 ppm or less, 30 ppm or less, or 25 ppm or less.

When a formyl group remains on the insoluble base material as a carrier, a compound other than a target compound may be non-specifically adsorbed on the remaining formyl group so that it may become impossible to selectively adsorb the target compound only. The above-described complex of Lewis base and borane is used for reducing the imine and inactivating the remaining formyl group which is not reacted with the ligand in order to reduce the above-described non-specific adsorption.

The above pKₐ may be 6.4 or less, 6.3 or less, or 6.2 or less. On the one hand, the lower limit of the pKₐ is not particularly restricted. When a borane complex having Lewis base of which pKₐ is lower is used, an amount of the ligand on the adsorbent may tend to be reduced; and when pKₐ is excessively low, it may difficult to form a complex with borane. The pKₐ may be 0.2 or more, not less than 0.5 or 1.0, or not less than 2.0, 3.0 or 4.0, and may be 5.0 or more.

The Lewis base usable in one or more embodiments of the present invention is a compound of which electron pair can be donated to borane to form a complex, of which pKₐ is 6.5 or less and which is excellent in reduction action to the imino group of the imine.

The Lewis base usable in one or more embodiments of the present invention is exemplified by amine, phosphine, phenol, amide, urea and oxime of which pKₐ are 6.5 or less.

The pKₐ tends to decrease in the case where an unshared electron pair of a nitrogen atom is conjugate to an aromatic ring. As an amine of which pKₐ is 6.5 or lower and which is usable in one or more embodiments of the present invention, a nitrogen-containing heterocyclic aromatic compound and/or an aromatic hydrocarbon compound having an amino group is exemplified.

The “nitrogen-containing heterocyclic aromatic compound” usable in one or more embodiments of the present invention means an aromatic compound which has at least one nitrogen atom in the aromatic ring and of which pKₐ value is 6.5 or less, and is exemplified by a five-membered nitrogen-containing heterocyclic aromatic compound such as pyrrol; a six-membered nitrogen-containing heterocyclic aromatic compound such as pyridine, pyridazine, pyrimidine and pyrazine; and a condensed nitrogen-containing heterocyclic aromatic compound such as quinoline, isoquinoline, phthalazine, quinazoline and quinoxaline.

The “aromatic hydrocarbon compound having an amino group” means an aromatic hydrocarbon compound of which aromatic ring has one or more directly bound amino groups as substituent groups and of which pKₐ value is 6.5 or less. The amino group is exemplified by —NH₂, a mono(C₁₋₆ alkyl)amino group and a di(C₁₋₆ alkyl)amino group. The number of an amino group as a substituent group may be 1 or 2, since the pKₐ value tends to become larger in the case of larger substitution number. The aromatic hydrocarbon compound is exemplified by a C₆₋₁₂ aromatic hydrocarbon compound such as benzene, naphthalene and biphenyl.

The nitrogen-containing heterocyclic aromatic compound may have a substituent group such as an amino group as long as the pKₐ value is 6.5 or less, and the aromatic hydrocarbon compound may be have a substituent group other than an amino group as long as the pKₐ value is 6.5 or less. A substituent group other than an amino group is exemplified by one or more substituent groups selected from the group essentially consisting of a C₁₋₆ alkyl group, a C₁₋₆ alkoxy group, a hydroxy group, a halogen atom, a cyano group and a nitro group.

In fact, an amine of which pKₐ value is described as 6.5 or less in an information document or of which pKₐ values actually measured is 6.5 or less may be selected, since a pKₐ value is changed depending on a kind and the number of a substituent group. The above-described nitrogen-containing heterocyclic aromatic compound and aromatic hydrocarbon compound are exemplified by pyridine; picoline such as α-picoline, β-picoline and γ-picoline; diphenylamine; toluidine such as o-toluidine, m-toluidine and p-toluidine; and pyrrol. The present invention is not restricted to the examples.

A pKₐ value of an aliphatic amine may become 6.5 or less in some cases depending on a kind and the number of a substituent group. An aliphatic amine having a pKₐ value of 6.5 or less is exemplified by a hydroxylamine and an alkoxyamine, such as hydroxylamine, methylhydroxylamine, N-methylhydroxylamine and N,O-dimethylhydroxylamine; and a cyano(C₁₋₆ alkyl)amine such as cyanomethyldiethylamine, di(cyanomethyl)amine and di(cyanoethyl)amine.

A phosphine having a pKₐ value of 6.5 or less is exemplified by a tertiary phosphine having an electron-withdrawing group, a secondary phosphine and a primary phosphine. The tertiary phosphine having an electron-withdrawing group is exemplified by a 2-cyanoethyl-di(C₁₋₆ alkyl)phosphine, a phenyl-di(C₁₋₆ alkyl)phosphine, a di(2-cyanoethyl) (C₁₋₆ alkyl)phosphine, triphenylphosphine and tri(2-cyanoethyl)phosphine. The secondary phosphine is exemplified by a di(C₁₋₆ alkyl)phosphine, diphenylphosphine and di(2-cyanoethyl)phosphine. The primary phosphine is exemplified by a (C₁₋₆ alkyl)phosphine.

A phenol having a pKₐ value of 6.5 or less is exemplified by a phenol having an electron-withdrawing substituent group at o-position or p-position, specifically 2,4-dinitrophenol, 2-chlorophenol, 2-bromophenol and 4-nitrophenol.

An amide having a pKₐ value of 6.5 or less is exemplified by cyanamide, a (C₁₋₆ alkyl) cyanamide and acetamide.

An urea having a pKₐ value of 6.5 or less is exemplified by urea, nitrourea and thiourea.

An oxime having a pKₐ value of 6.5 or less is exemplified by oxamide oxime, benzamidoxime, α-phenylacetamideoxime, succinamidoxime and toluamideoxime.

The above-described borane complex can be generally produced by reacting diborane produced from sodium borohydride and a Lewis base ligand.

As a solvent for the reduction reaction, an aqueous solvent may be used. The aqueous solvent is exemplified by water; an aqueous solution such as a buffer solution; a water-miscible organic solvent; a mixed solvent of an aqueous solution and a water-miscible organic solvent. A water-miscible organic solvent means an organic solvent which can be unlimitedly mixed with water, and is exemplified by a lower alcohol solvent such as methanol, ethanol and isopropanol; an amino solvent such as dimethylformamide and dimethylacetamide; and a sulfoxide solvent such as dimethylsulfoxide.

When an aqueous solvent is used in the reduction reaction of the present step, a denaturation or alteration of the ligand to be immobilized can be suppressed in comparison with the case where an organic solvent is used. Since an amine-borane complex may be insoluble in water in some cases, an appropriate amount of a water-miscible organic solvent may be added depending on a water solubility of the amine-borane complex to be used. A concentration of a water-miscible organic solvent in the aqueous solvent may be, for example, 50 mass % or less, 25 mass % or less, or 10 mass % or less in some embodiments. The aqueous solvent may be water or an aqueous solution.

A pH of the reaction mixture for the reduction reaction of the present step may be 2 or more and less than 12 in some embodiments. When the pH is 2 or more, a degradation of the imino group and a deactivation of the amine-borane complex due to a reaction with water may be inhibited more surely. On the one hand, when the pH is less than 12, the reaction of the amine-borane complex may be further accelerated. The pH may be 5 or more, or 7 or more, and may be less than 10, or less than 9. The pH can be measured using a pH meter which is subjected to three-point calibration by using a standardized solutions having pH of 3 to 5, 6 to 7 and 9 to 10.

The condition such as reaction temperature and reaction temperature of the present step may be adjusted to those for the above-described step 3 of reacting the ligand and the insoluble base material.

After the reaction, reagents other than the ligand covalently immobilized on the insoluble base material may be removed by washing the adsorbent according to one or more embodiments of the present invention method. A washing liquid and a washing method are not particularly restricted, and may include passing a solution or adding a solution and stirring the mixture. In one or more embodiments, a solution may include at least one of water, acetic acid, an organic solvent such as an alcohol, a liquid having pH of 2 or more and 5 or less, a liquid having pH of 8 or more and 13 or less, sodium chloride, potassium chloride, sodium acetate, disodium hydrogenphosphate, sodium dihydrogenphosphate, a buffering agent, a surfactant, urea, guanidine, guanidine hydrochloride and other regenerant. In one or more embodiments, the adsorbent may be washed more than once by using the same or different solutions.

An amount of the ligand introduced into the adsorbent of one or more embodiments of the present invention per 1 mL of the formyl group-containing insoluble base material may be 1 mg or more and 500 mg or less. When the amount of the introduced ligand per 1 mL of the formyl group-containing insoluble base material is 1 mg or more, an amount of the adsorbed target amount may become large in some embodiments. When the amount is 500 mg or less, a production cost may be reduced in some embodiments. In one or more embodiments, the amount of the introduced ligand per 1 mL of the formyl group-containing insoluble base material may be 2 mg or more, 3 mg or more, or 4 mg or more, and may be 120 mg or less, 60 mg or less, 30 mg or less, or 20 mg or less. A volume of the adsorbent as a base of the introduced ligand amount is a volume of a whole structure containing a hole and a skeleton in the case of a monolith and a porous membrane, or is a tapping volume in the case of porous beads, similarly to a volume of the formyl group-containing insoluble base material as a base of formyl group amount.

In addition, an amount of the ligand introduced into the adsorbent according to one or more embodiments of the present invention per 1 mL of the formyl group-containing insoluble base material may be 0.01 μmol or more and 15 μmol or less in some embodiments. When the amount of the introduced ligand per 1 mL of the formyl group-containing insoluble base material is 0.01 μmol or more, an amount of the adsorbed target amount may become large in some embodiments. When the amount is 15 μmol or less, a production cost may be reduced in some embodiments. The amount of the introduced ligand per 1 mL of the formyl group-containing insoluble base material may be 0.03 μmol or more, 0.05 μmol or more, or 0.1 μmol or more, and may be 5 μmol or less, 2 μmol or less, 0.75 μmol or less, or 0.5 μmol or less.

In one or more embodiments, an amount of the introduced ligand can be determined by measuring an absorbance due to the affinity ligand in a supernatant of the reaction mixture before and after the immobilization reaction and calculating an amount of the unreacted ligand on the basis of the difference of the measured values from the assumption that all of the ligand other than the unreacted ligand binds to the insoluble base material. In some embodiments, the amount of the introduced ligand can be determined by an element analysis method. For example, in the case of an amino group-containing ligand, the amount of the introduced ligand can be determined by analyzing an amount of N contained in the adsorbent.

5. Use Application Example of Adsorbent

When the adsorbent produced by firmly immobilizing the ligand on the insoluble base material according to one or more embodiments of the present invention method described above is used for purifying a target compound, leakage of the ligand may be suppressed, which may prevent mixing of the ligand and the target compound.

In order to purify a target compound by using the adsorbent according to one or more embodiments of the present invention, a mixed liquid containing the target compound is contacted with the adsorbent. The contact method is not particularly restricted, and for example, merely the adsorbent may be added in the mixed liquid and the mixture may be mixed, but it is efficient and convenient that a column is filled with the adsorbent and the mixed liquid is passed through the column.

For example, a column having a diameter of 0.1 cm or more and 2000 cm or less and a height of 1 cm or more and 5000 cm or less may be used in some embodiments. When the diameter is 0.1 cm or more and the height is 1 cm or more, a target compound may be efficiently adsorbed in some embodiments. In some embodiments, the diameter may be 2000 cm or less and the height may be 5000 cm or less.

A residence time to contact the adsorbent with the mixed liquid containing a target compound may be 1 minute or more in terms of an accuracy of the adsorption and a durability of a device in some embodiments. In some embodiments, the residence time may be 12 minutes or less in terms of an efficiency. In some embodiments, the residence time may be 2 minutes or more, or 3 minutes or more, and may be 10 minutes or less, or 9 minutes or less.

In one or more embodiments, a specific adsorption condition is adjusted so that, for example, an amount of an adsorbed target compound per 1 mL of the adsorbent becomes 1 mg or more. When the adsorption amount is 1 mg or more, the target compound may be efficiently purified. In some embodiments, when the adsorption amount is 200 mg or less, the adsorbed target compound may be easily eluted. In some embodiments, the adsorption amount may be 10 mg or more and 150 mg or less, 20 mg or more and 100 mg or less, 30 mg or more and 90 mg or less, or 40 mg or more and 80 mg or less.

A method for determining an amount of an adsorbed target compound is not particularly restricted, and the amount can be determined as a static adsorption amount and a dynamic adsorption amount. For example, a static adsorption amount can be determined by contacting a solution prepared by dissolving 70 mg of a target compound in 35 mL of a phosphate buffer having pH of 7.4 with 0.5 mL of the adsorbent sufficiently washed with the phosphate buffer having pH of 7.4, stirring the mixture at 25° C. for 2 hours, and measuring an amount of the reduced target compound in a supernatant.

After a target compound is adsorbed on the adsorbent according to one or more embodiments of the present invention, the adsorbent may be washed to remove a non-specific adsorbed substance. A washing condition is not particularly restricted, and an absorbent may be washed in some embodiments with a buffer solution having a pH of 6.0 or more and 8.0 or less, ultrapure water, pure water, reverse osmosis water, or distilled water, so that the adsorbed target compound is not detached.

In one or more embodiments, a purified target compound can be obtained by desorbing the target compound adsorbed on the adsorbent. In order to desorb a target compound from the adsorbent, for example, the adsorbent may be washed with a buffer solution having a pH of 3.0 or more and 5.0 or less.

The present application claims the benefit of the priority date of Japanese patent application No. 2015-168066 filed on Aug. 27, 2015. All of the contents of the Japanese patent application No. 2015-168066 filed on Aug. 27, 2015, are incorporated by reference herein.

### EXAMPLES

Hereinafter, the examples are described to demonstrate one or more embodiments of the present invention more specifically, but the present invention is in no way restricted by the examples, and the examples can be appropriately modified to be carried out within a range which adapts to the contents of this specification. Such a modified example is also included in the scope of the present invention disclosure.

### Example 1: Preparation of Adsorbent Using Borane-Pyridine Complex

As an insoluble base material, crystalline highly crosslinked cellulose (manufactured by JNC CORPORATION, gel produced by the method described in JP 2009-242770 A) was used. The insoluble base material (3.5 mL) was sufficiently washed on a glass filter using a 0.01 M citrate buffer of pH 3 prepared using trisodium citrate dihydrate, citric acid monohydrate and reverse osmosis water. Then, the washed insoluble base material was added into a centrifuge tube, and the citrate buffer was added thereto so that the total liquid amount became 6 mL. An aqueous solution prepared by dissolving 22.5 mg of sodium periodate in 2 mL of reverse osmosis water was added thereto, and the mixture was shaken at 6° C. for about 30 minutes using a mix rotor to oxidize 1,2-diol group of the insoluble base material to be a formyl group. The mixture was subjected to filtration using a glass filter, and the formyl group-containing carrier was sufficiently washed with reverse osmosis water to obtain a formyl group-containing carrier.

On the glass filter, 3.5 mL of the obtained formyl group-containing carrier was sufficiently washed with 0.6M citrate buffer of pH 12 prepared from trisodium citrate dihydrate, sodium hydroxide and reverse osmosis water. Then, the washed formyl group-containing carrier was added into a centrifuge tube, and the citrate buffer was added thereto so that the total liquid amount became 7.5 mL. Separately, Protein A having the amino acid sequence of SEQ ID NO: 2 described in WO 2011/118699 was prepared with reference to the international publication. To the formyl group-containing carrier, 0.91 mL of 57.98 mg/mL Protein A aqueous solution was added. The mixture was shaken at 6° C. for 23 hours using a mix rotor. Then, 2.4 M citric acid aqueous solution prepared from citric acid monohydrate and reverse osmosis water was added thereto so that the pH of the mixture was adjusted to 8, and the mixture was continuously shaken at 6° C. for 4 hours. To the mixture, 200 μL of borane-pyridine complex manufactured by Tokyo Chemical Industry Co., Ltd. was added. The mixture was shaken at 25° C. for 18 hours. After the reaction, an amount of the ligand immobilized on the carrier was determined by measuring an absorbance of a maximum absorption of the reaction mixture at 276 nm. As a result, the immobilized ligand amount was 14 mg/mL-gel.

The obtained carrier was washed on a glass filter with reverse osmosis water until an electrical conductivity of a washing filtrate became 5 μS/cm or less, and further washed with 0.1 M citric acid aqueous solution, a mixed aqueous solution of 0.05 M sodium hydroxide and 0.5 M sodium sulfate, and a citrate buffer which contained 0.5 M trisodium citrate dihydrate and citric acid monohydrate and of which pH was 6 sequentially. At last, the adsorbent was washed with reverse osmosis water until an electrical conductivity of a washing filtrate became 5 μS/cm or less to obtain a ligand-immobilized adsorbent.

### Example 2: Preparation of Adsorbent Using Borane-Picoline Complex

A ligand-immobilized adsorbent was obtained similarly to Example 1 except that 3.5 mL of 5.5 mass % ethanol solution of borane-α-picoline complex manufactured by SIGMA ALDRICH was used in place of borane-pyridine complex as a reducing agent.

### Comparative Example 1: Preparation of Adsorbent Using Borane-Dimethylamine Complex

A ligand-immobilized adsorbent was obtained similarly to Example 1 except that 1.93 mL of 5.5 mass % reverse osmosis water solution of borane-dimethylamine complex manufactured by Wako Pure Chemical Industries, Ltd. was used in place of borane-pyridine complex as a reducing agent.

### Comparative Example 2: Preparation of Adsorbent Using Borane-Trimethylamine Complex

A ligand-immobilized adsorbent was obtained similarly to Example 1 except that 4.78 mL of 5.5 mass % reverse osmosis water solution of borane-trimethylamine complex manufactured by Tokyo Chemical Industry Co., Ltd. was used in place of borane-pyridine complex as a reducing agent.

### Comparative Example 3: Preparation of Adsorbent Using Borane-N,N-diethylaniline Complex

A ligand-immobilized adsorbent was obtained similarly to Example 1 except that 200 μL of borane-N,N-diethylaniline complex manufactured by Tokyo Chemical Industry Co., Ltd. was used in place of borane-pyridine complex as a reducing agent.

### Test Example 1: Determining Amount of Leaked Ligand

A human IgG was adsorbed on the ligand-immobilized adsorbent prepared by the above-described Examples and Comparative examples, and an amount of a leaked ligand was determined.

(1) Preparation of Solutions

The following Liquids A to E and Neutralizing liquid were prepared and deaerated before use.

Liquid A: phosphate buffer solution having a pH of 7.4, prepared from Phosphate buffered saline manufactured by SIGMA and reverse osmosis water

Liquid B: 35 mM sodium acetate aqueous solution having a pH of 3.5, prepared from acetic acid, sodium acetate and reverse osmosis water

Liquid C: 1 M acetic acid aqueous solution prepared from acetic acid and reverse osmosis water

Liquid D: IgG aqueous solution having a concentration of 3 mg/mL, prepared from polyclonal antibody (“Gammagard” manufactured by Baxter) and the above-described Liquid A

Liquid E: mixed aqueous solution of 0.1 M sodium hydroxide and 1 M sodium chloride, prepared from sodium hydroxide, sodium chloride and reverse osmosis water

Neutralizing liquid: 2 M tris(hydroxymethyl)aminomethane aqueous solution prepared from tris(hydroxymethyl)aminomethane and reverse osmosis water

(2) Filling and Preparation

Into a column having a diameter of 0.5 cm and a height of 15 cm in a column chromatography apparatus (“AKTAexplorer100” manufactured by GE Healthcare), 3 mL as a sedimentation volume of the adsorbent sample prepared in the above-described Examples and Comparative examples was added. The column was filled with the adsorbent sample by flowing 0.2 M reverse osmosis water solution of sodium chloride at a linear speed of 230 cm/h for 15 minutes. On a fraction collector, 15 mL correcting tubes were set. In the correcting tubes for an eluent, Neutralizing liquid was preliminarily added.

(3) Purification of IgG

Through the above-described column, 15 mL of Liquid A was flowed and then 100 mL of Liquid D, i.e. IgG aqueous solution, was flowed. Next, after 21 mL of Liquid A was flowed, 12 mL of Liquid B was flowed to elute IgG. Then, 6 mL of Liquid C, 6 mL of Liquid E and 15 mL of Liquid A were flowed. The flow speed of each liquid was adjusted to 0.5 mL/min or 1 mL/min so that the residence time to be contacted with the adsorbent was 6 minutes or 3 minutes.

(4) Determining Amount of Leaked Ligand

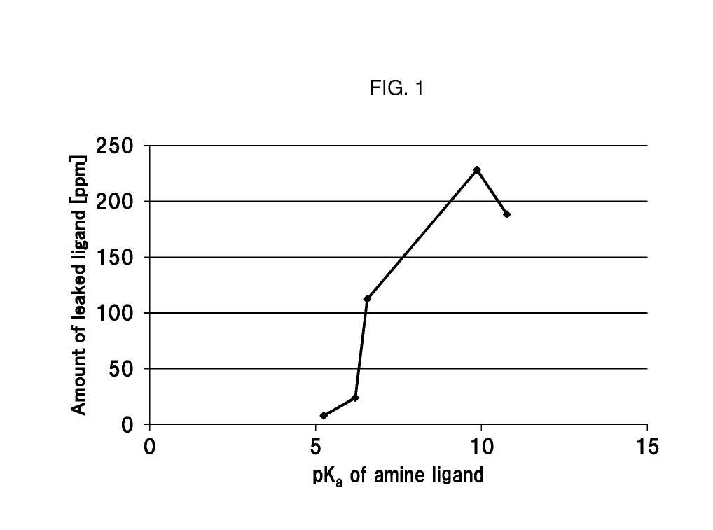

In order to evaluate an amount of a leaked ligand, an amount of the ligand contained in the eluted IgG solution was measured. Specifically, the eluate obtained in the above-described (3) was recovered, amounts of IgG and ligand (Protein A) in the eluate were measured, and a concentration of the ligand leaked in the purified IgG was determined as a leakage amount. The ligand concentration was measured by ELISA method. The relation between pKₐ of the amine ligand in the borane complex and the amount of the leaked ligand is shown in Table 1 and FIG. 1.

TABLE 1

| | | pKₐ of | Amount of |
|---|---|---|---|
| | Amine ligand | amine ligand | leaked ligand |
| Example 1 | pyridine | 5.3 | 8 ppm |
| Example 2 | α-picoline | 6.2 | 24 ppm |
| Comparative | dimethylamine | 10.8 | 188 ppm |
| example 1 | | | |
| Comparative | trimethylamine | 9.8 | 228 ppm |
| example 2 | | | |
| Comparative | N,N-diethylaniline | 6.6 | 112 ppm |
| example 3 | | | |

As the result shown in Table 1 and FIG. 1, while the pKₐ value of the amine which coordinates with borane in the borane complex is changed from 6.2 to 6.6, an amount of the leaked ligand is significantly increased. On the one hand, when the pKₐ value is 6.5 or less, an amount of the leaked ligand is remarkably suppressed. When the pKₐ value is 6.2 or less, an amount of the leaked ligand is further reduced. Thus, it was demonstrated that an amount of a leaked ligand can be remarkably reduced by reducing an imino group with the borane complex which is coordinated with amine having the pKₐ value of 6.5 or less to immobilize a ligand on an insoluble base material.

### Example 3: Preparation of Agarose Adsorbent Using Borane-Picoline Complex

An adsorbent on which the ligand was immobilized in a ratio of 14 mg/mL was obtained similarly to Example 1 except that 3.5 mL of a formyl group-containing agarose base material (“High Density glyoxal” manufactured by ABT) was used as a formyl group-containing carrier.

### Example 4: Preparation of Synthetic Polymer Adsorbent Using Borane-Picoline Complex

An adsorbent on which the ligand was immobilized in a ratio of 14 mg/mL was obtained similarly to Example 1 except that 3.5 mL of a formyl group-containing synthetic polymer base material (“toyopearl AF-Formyl 650M” manufactured by Tosoh Corporation) was used as a formyl group-containing carrier.

### Example 5: Preparation of Glass Adsorbent Using Borane-Picoline Complex

To a mixed solution of 10 mL of special grade toluene manufactured by Wako Pure Chemical Industries, Ltd. and 1 mL of 3-glycidoxypropyl-trimethoxysilane manufactured by Shin-Etsu Chemical Co., Ltd., 8 mL of glass beads (“Silica gel 60” manufactured by Millipore) was added as an insoluble base material. The mixture was stirred at 90° C. for 8 hours. Then, the beads were washed with acetone and reverse osmosis water to obtain an epoxy group-introduced carrier. The epoxy group-introduced carrier was added to 0.2 N sulfuric acid. The ring of the epoxy group was opened by stirring the mixture at 50° C. for 3 hours to obtain a diol group-introduced carrier. An adsorbent on which the ligand was immobilized in a ratio of 4 mg/mL was obtained similarly to Example 1 except that the diol group-introduced carrier was used and the pH for immobilizing the ligand was adjusted to 9.

### Example 6: Preparation of Protein G-Immobilized Carrier Using Borane-Picoline Complex

An adsorbent on which the ligand was immobilized in a ratio of 15 mg/mL was obtained similarly to Example 1 except that Protein G manufactured by Prospec was used as a ligand.

### Example 7: Preparation of Protein L-Immobilized Carrier Using Borane-Picoline Complex

An adsorbent on which the ligand was immobilized in a ratio of 14 mg/mL was obtained similarly to Example 1 except that Protein L manufactured by Prospec was used as a ligand.

### Comparative Example 4: Preparation of Agarose Adsorbent Using Borane-Dimethylamine Complex

An adsorbent on which the ligand was immobilized in a ratio of 15 mg/mL was obtained similarly to Example 3 except that 1.93 mL of 5.5 mass % reverse osmosis water solution of borane-dimethylamine complex manufactured by Wako Pure Chemical Industries, Ltd. was used in place of borane-picoline complex as a reducing agent.

### Comparative Example 5: Preparation of Synthetic Polymer Adsorbent Using Borane-Dimethylamine Complex

An adsorbent on which the ligand was immobilized in a ratio of 14 mg/mL was obtained similarly to Example 4 except that 1.93 mL of 5.5 mass % reverse osmosis water solution of borane-dimethylamine complex manufactured by Wako Pure Chemical Industries, Ltd. was used in place of borane-picoline complex as a reducing agent.

### Comparative Example 6: Preparation of Glass Adsorbent Using Borane-Dimethylamine Complex

An adsorbent on which the ligand was immobilized in a ratio of 4 mg/mL was obtained similarly to Example 5 except that 1.93 mL of 5.5 mass % reverse osmosis water solution of borane-dimethylamine complex manufactured by Wako Pure Chemical Industries, Ltd. was used in place of borane-picoline complex as a reducing agent.

### Comparative Example 7: Preparation of Protein G-Immobilized Carrier Using Borane-Dimethylamine Complex

An adsorbent on which the ligand was immobilized in a ratio of 15 mg/mL was obtained similarly to Example 6 except that 1.93 mL of 5.5 mass % reverse osmosis water solution of borane-dimethylamine complex manufactured by Wako Pure Chemical Industries, Ltd. was used in place of borane-picoline complex as a reducing agent.

### Comparative Example 8: Preparation of Protein L-Immobilized Carrier Using Borane-Dimethylamine Complex

An adsorbent on which the ligand was immobilized in a ratio of 14 mg/mL was obtained similarly to Example 7 except that 1.93 mL of 5.5 mass % reverse osmosis water solution of borane-dimethylamine complex manufactured by Wako Pure Chemical Industries, Ltd. was used in place of borane-picoline complex as a reducing agent.

### Test Example 2: Determining Amount of Leaked Ligand

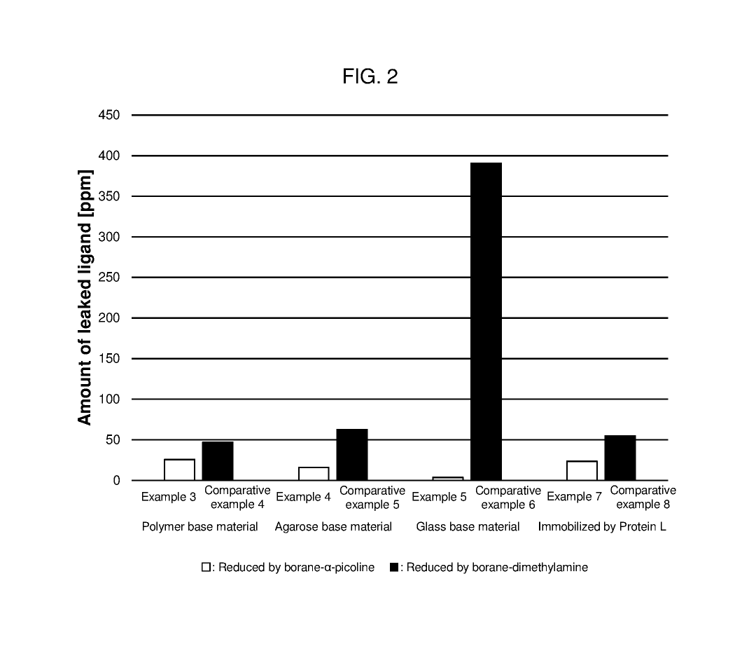

An amount of the leaked ligand was determined similarly to the above-described Test example 1 except that the ligand-immobilized adsorbent prepared in Examples 3 to 5, 7 and Comparative Examples 4 to 6, 8 was used. The result is shown in FIG. 2.

As the result shown in FIG. 2, it was demonstrated that an amount of a leaked ligand can be remarkably reduced by reducing an imino group with the borane complex which is coordinated with amine having the pKₐ value of 6.2 or less to immobilize a ligand on an insoluble base, even when a polymer base material, agarose base material or silica base material is used as an insoluble base material and even when Protein L is used as a ligand.

Although embodiments of the disclosure have been described using specific terms, devices, and methods, such description is for illustrative purposes only. In the present disclosure, lower and upper limits may be used alone to define an embodiment or may be combined to provide a range of values. The words used are words of description rather than limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. Therefore, the spirit and scope of the appended claims should not be limited to the description of the preferred versions contained therein.

1. A method for immobilizing a ligand on a formyl group-containing insoluble base material, the method comprising:
producing an imine by mixing the ligand and the formyl group-containing insoluble base material; and
reducing the imine by using a borane complex,
wherein the ligand comprises an amino group and has a specific affinity for a target compound, and
wherein the borane complex has a Lewis base ligand having pKₐ of 6.5 or less.

2. The method according to claim 1, wherein the Lewis base ligand is one or more selected from the group consisting of a nitrogen-containing heterocyclic aromatic compound, and an aromatic hydrocarbon compound having an amino group as a substituent.

3. The method according to claim 1, wherein the ligand is a peptide.

4. The method according to claim 3, wherein the ligand is an antibody affinity ligand.

5. The method according to claim 4, wherein the antibody affinity ligand is Protein A, Protein G, Protein L or an analog thereof.

6. The method according to claim 1, wherein the formyl group-containing insoluble base material is at least one selected from the group consisting of a polysaccharide, a synthetic polymer and glass.

7. The method according to claim 1, wherein the formyl group-containing insoluble base material is cellulose or agarose.

8. The method according to any one of claim 1, wherein a form of the formyl group-containing insoluble base material is a porous bead, a monolith or a porous membrane.

9. A method for purifying a target compound, comprising:
producing an adsorbent by immobilizing a ligand on a formyl group-containing insoluble base material by the method according to claim 1; and
contacting a mixture containing the target compound with the adsorbent to adsorb the target compound on the adsorbent.
